(19) United States
(12) Patent Application Publication
(10) Pub. No.: US 2026/0192257 A1
(43) Pub. Date: Jul. 9, 2026

(54) METHOD FOR EVALUATING OXIDATION RISK OF SEPARATION MEMBRANE, PROGRAM FOR EVALUATING OXIDATION RISK, RECORDING MEDIUM, AND EVALUATION DEVICE

(71) Applicant: Toray Industries, Inc., Chuo-ku, Tokyo (JP)

(72) Inventors: Yoshimi ISHIKAWA, Iyo-gun, Ehime (JP); Tomohiro MAEDA, Otsu-shi, Shiga (JP); Koji NAKATSUJI, Iyo-gun, Ehime (JP)

(73) Assignee: Toray Industries, Inc., Chuo-ku, Tokyo (JP)

(21) Appl. No.: 19/131,591

(22) PCT Filed: Nov. 22, 2023
(86) PCT No.: PCT/JP2023/041997

§ 371 Date: May 21, 2025

(30) Foreign Application Priority Data
Nov. 25, 2022 (JP) 2022-188059

(51) Int. Cl. B01D 65/10; G01N 27/416

(52) U.S. Cl. CPC B01D 65/10; G01N 27/4168

(57) ABSTRACT

The present invention relates to an oxidation risk evaluation method of a separation membrane in a water treatment plant, including: collecting a deposit on a used separation membrane; bringing the deposit into contact with a solution containing a sulfite or bisulfite; and evaluating an oxidation potential of the deposit based on a generated oxidative substance.

### CROSS REFERENCE TO RELATED APPLICATIONS

This application is the U.S. National Phase of PCT/JP2023/041997, filed Nov. 22, 2023, which claims priority to Japanese Patent Application No. 2022-188059, filed Nov. 25, 2022, the disclosures of these applications being incorporated herein by reference in their entireties for all purposes.

### FIELD OF THE INVENTION

The present invention relates to an oxidation risk evaluation method and an oxidation risk evaluation program of a separation membrane, a recording medium, and an evaluation device.

### BACKGROUND OF THE INVENTION

In recent years, a water treatment technique using various separation membranes such as a gas separation membrane, a reverse osmosis membrane, a nanofiltration membrane, an ultrafiltration membrane, and a microfiltration membrane has attracted attention as a highly accurate and energy-saving treatment process, and is being applied to various water treatments. For example, in a reverse osmosis separation method using a reverse osmosis membrane, it is possible to obtain a liquid in which a concentration of a solute such as salt is reduced by allowing a solution containing the solute such as salt to permeate the reverse osmosis membrane at a pressure equal to or higher than an osmotic pressure of the solution, and the reverse osmosis separation method is widely used for desalination of seawater or brine, production of ultra-pure water, concentration and recovery of valuable resources, and the like, and forms a core of a water treatment membrane separation technique.

Main problems when the reverse osmosis membrane is applied to the above application include surface contamination of a semipermeable membrane called fouling and chemical deterioration of the semipermeable membrane. One method to prevent the former is, for example, a method of adding an oxidative substance such as sodium hypochlorite to sterilize microorganisms. However, the oxidative substance contained in such a pretreatment contributes to oxidation degradation. As an oxidative substance detection method, an oxidation-reduction potential (ORP) measurement, an N, N-diethyl para phenylenediamine (DPD) method which is a color reaction, a Fujiwara test described in Patent Literature 1 and Non Patent Literature 1, and the like are used. On the other hand, examples of a substance that is not an oxidative substance but plays an important role in an oxidation reaction process include oxidation reaction promoting substances represented by certain transition metals. An amount of the substance is difficult to evaluate by the oxidative substance detection method described above, and another method includes, for example, a method of examining elemental composition of a feed water-containing substance by high-frequency inductively coupled plasma (ICP) emission spectrometry or the like. However, this spectroscopic analysis method requires advanced and expensive equipment, requires a specialized analysis institution, and takes time and high cost to obtain a result. When an amount of an oxidation reaction promoting substance contained in feed water is equal to or less than a detection lower limit value of the spectroscopic analysis method, it is difficult to evaluate an oxidation risk. On the other hand, Patent Literature 2 discloses a method of evaluating an oxidation risk of a separation membrane in a water treatment plant by passing membrane filtration feed water through an oxidative substance-sensitive member. However, when the amount of the oxidation reaction promoting substance contained in the feed water is small, there is a problem that the detection is difficult for this method.

### PATENT LITERATURE

Patent Literature 1: JP2020-6322A
Patent Literature 2: JP2016-190212A
Patent Literature 3: JP2021-6335A

### Non-Patent Literature

Non Patent Literature 1: Journal of Membrane Science, 2010, Vol. 347, P. 159-164.

### SUMMARY OF THE INVENTION

The present invention can simply and quickly evaluate an oxidation risk, which is difficult to evaluate by the method in the related art, by evaluating an oxidation potential using a deposit on a surface of a separation membrane. In addition, the present invention can be easily and quickly implemented and a small amount of the deposit is required, therefore the present invention is useful for avoiding a risk and quickly resolving an operational problem in a water treatment plant.

The present invention is an oxidation risk evaluation method including: collecting a deposit on a surface of a separation membrane; bringing the deposit into contact with a solution containing a sulfite or bisulfite; and evaluating an oxidation potential of the deposit based on a generated oxidative substance.

In order to solve the above problems, the present invention has the following features.

(1) An oxidation risk evaluation method of a separation membrane in a water treatment plant, including:
collecting a deposit on a used separation membrane;
bringing the deposit into contact with a solution containing a sulfite or bisulfite; and
evaluating an oxidation potential of the deposit based on a generated oxidative substance.

(2) The oxidation risk evaluation method of a separation membrane according to (1), in which a deposit extracted liquid is used instead of the deposit.

(3) The oxidation risk evaluation method of a separation membrane according to (1) or (2), in which the separation membrane is any one of the followings:
A: a separation membrane taken from a semipermeable membrane element that separates a feed water into a permeate water and a concentrate water in a water treatment plant,
B: a separation membrane provided inside a water passing member installed in a feed water line of a water treatment plant, and
C: a separation membrane provided inside a water passing member installed in a concentrate water line in a water treatment plant in which a semipermeable membrane element separating a feed water into a permeate water and a concentrate water is used.

(4) The oxidation risk evaluation method of a separation membrane according to any one of (1) to (3), in which the oxidation risk evaluation method of a separation membrane is an oxidation risk evaluation method performed by a catalytic reaction, and the method of evaluating the oxidation potential of the deposit is a method of measuring a change over time in an oxidation power index value of the solution brought into contact with the deposit, and any one of the followings is evaluated:
A: a maximum value of the oxidation power index value,
B: a change rate of the oxidation power index value per unit time,
C: a maximum value of a moving average value of the oxidation power index value, and
D: a change rate of a moving average value of the oxidation power index value per unit time.

(5) The oxidation risk evaluation method of a separation membrane according to any one of (1) to (4), in which the method of evaluating the oxidation potential of the deposit is a method of adding a sulfite or bisulfite to the solution brought into contact with the deposit to change a sulfite or bisulfite concentration of the solution, and measuring an oxidation power index value when the deposit is brought into contact with the solution.

(6) The oxidation risk evaluation method of a separation membrane according to any one of (1) to (5), in which in the method of evaluating the oxidation potential of the deposit, the solution brought into contact with the deposit is a sulfite or bisulfite solution and two or more types of the solution with different concentrations are used, and the oxidation power index value of the solution brought into contact with the deposit is measured.

(7) The oxidation risk evaluation method of a separation membrane according to (5) or (6), in which the method of evaluating the oxidation potential of the deposit according to (5) or (6) is an evaluation of at least any one of the followings:
A: a maximum value of the oxidation power index value, and
B: a change rate of the oxidation power index value per unit sulfite or bisulfite concentration.

(8) The oxidation risk evaluation method of a separation membrane according to any one of (5) to (7), which is a method of measuring the oxidation power index value when the sulfite or bisulfite is further added after the oxidation power index value becomes maximum.

(9) The oxidation risk evaluation method of a separation membrane according to any one of (5) to (7), which is a method of measuring the oxidation power index value when a different solution with a higher concentration is further used to come into contact with the deposit after the oxidation power index value becomes maximum.

(10) The oxidation risk evaluation method of a separation membrane according to (7) or (8), in which the method of evaluating the oxidation potential of the deposit according to (7) or (8) is a method of evaluating at least any one of the followings:
A: a sulfite or bisulfite concentration at which the oxidation power index value is equal to or less than the oxidation power index value before contact with the deposit, and
B: a change rate of the oxidation power index value per unit sulfite or bisulfite concentration.

(11) The oxidation risk evaluation method of a separation membrane according to any one of (1) to (10), in which the deposit is brought into contact with the solution containing the sulfite or bisulfite having a hydrogen ion concentration index (pH) of 9.0 or more.

(12) An oxidation risk evaluation program of a separation membrane in a water treatment plant, which utilizes the oxidation risk evaluation method according to (1), in which
in order to evaluate an oxidation risk, the program causes a computer to function as a data recording unit configured to record input data in the computer and as a unit configured to evaluate the oxidation potential of the deposit based on the recorded data, and the input data includes a condition and an oxidation power index value when the deposit collected from the used separation membrane is brought into contact with the solution containing the sulfite or bisulfite.

(13) The oxidation risk evaluation program according to (12), in which the oxidation risk evaluation method of a water treatment plant is an oxidation risk evaluation method performed by a catalytic reaction, and the unit configured to evaluate the oxidation potential of the deposit is a unit configured to evaluate, based on a data over time of the oxidation power index value, at least any one of the followings:
A: a maximum value of the oxidation power index value,
B: a change rate of the oxidation power index value per unit time,
C: a maximum value of a moving average value of the oxidation power index value, and
D: a change rate of a moving average value of the oxidation power index value per unit time.

(14) The oxidation risk evaluation program according to (12), in which the unit configured to evaluate the oxidation potential of the deposit is a unit configured to evaluate, based on a relation of the oxidation power index value to a sulfite or bisulfite concentration, any one of the followings:
A: a maximum value of the oxidation power index value, and
B: a change rate of the oxidation power index value per unit sulfite or bisulfite concentration.

(15) The oxidation risk evaluation program according to (12), in which the unit configured to evaluate the oxidation potential of the deposit is a unit configured to evaluate, based on a relation of the oxidation power index value to a sulfite or bisulfite concentration after the oxidation power index value becomes maximum, any one of the followings:
A: a sulfite or bisulfite concentration at which the oxidation power index value is equal to or less than the oxidation power index value before contact with the deposit, and
B: a change rate of the oxidation power index value per unit sulfite or bisulfite concentration.

(16) A recording medium storing the oxidation risk evaluation program according to any one of (12) to (15).

(17) An oxidation risk evaluation device of a separation membrane in a water treatment plant, the oxidation risk evaluation device including:
a data input unit configured to input, to a computer, a condition and an oxidation power index value when a deposit collected from a used separation membrane is brought into contact with a solution containing a sulfite or bisulfite;
a data recording unit configured to record the condition and the oxidation power index value in the computer; and
a unit configured to evaluate an oxidation potential by the method according to any one of (1) to (11).

According to the present invention, it is possible to simply and quickly evaluate an oxidation risk of a separation membrane, which is difficult to determine by an evaluation method in the related art.

### BRIEF DESCRIPTION OF THE DRAWINGS

FIG. 1 shows an example of a flow of a water treatment plant.

FIG. 2 is a schematic view showing an example of a water passing member used in the present invention.

FIG. 3 shows an example of a separable water passing container (unit water passing member) constituting the water passing member used in the present invention.

FIG. 4 is a graph showing an example of a relation between an elapsed time from contact with a deposit and an oxidation power index value, and a method of evaluating a maximum value of the oxidation power index value in a first embodiment of evaluating an oxidation potential of the present invention.

FIG. 5 is a graph showing an example of the relation between the elapsed time from the contact with the deposit and the oxidation power index value, and a method of evaluating a change rate of the oxidation power index value per unit time in the first embodiment of evaluating the oxidation potential of the present invention.

FIG. 6 is a graph showing an example of a relation between a sulfite or bisulfite concentration and the oxidation power index value, and a method of evaluating the maximum value of the oxidation power index value in a second or third embodiment of evaluating the oxidation potential of the present invention.

FIG. 7 is a graph showing an example of the relation between the sulfite or bisulfite concentration and the oxidation power index value, and a method of evaluating a change rate of the oxidation power index value per unit sulfite or bisulfite concentration in the second or third embodiment of evaluating the oxidation potential of the present invention.

FIG. 8 is a graph showing an example of the relation between the sulfite or bisulfite concentration and the oxidation power index value, and a method of evaluating the sulfite or bisulfite concentration at which the oxidation power index value is equal to or less than that before contact with the deposit in a fourth or fifth embodiment of evaluating the oxidation potential of the present invention.

FIG. 9 is a graph showing an example of the relation between the sulfite or bisulfite concentration and the oxidation power index value, and a method of evaluating a change rate of the oxidation power index value per unit sulfite or bisulfite concentration in the fourth or fifth embodiment of evaluating the oxidation potential of the present invention.

FIG. 10 is an example of a program for causing a computer to execute an oxidation risk determination using the first embodiment of the present invention.

FIG. 11 is an example of the program for causing the computer to execute the oxidation risk determination using the second or third embodiment of the present invention.

FIG. 12 is an example of the program for causing the computer to execute the oxidation risk determination using the sulfite or bisulfite concentration at which the oxidation power index value is equal to or less than that before contact with the deposit in the fourth embodiment of the present invention.

FIG. 13 is an example of the program for causing the computer to execute the oxidation risk determination using the change rate of the oxidation power index value per unit sulfite or bisulfite in the fourth embodiment of the present invention.

FIG. 14 is an example of the program for causing the computer to execute the oxidation risk determination using the first embodiment of the present invention.

FIG. 15 is an example of the program for causing the computer to execute the oxidation risk determination using the second or third embodiment of the present invention.

FIG. 16 is an example of the program for causing the computer to execute the oxidation risk determination using the sulfite or bisulfite concentration at which the oxidation power index value is equal to or less than that before contact with the deposit in the fourth embodiment of the present invention.

FIG. 17 is an example of the program for causing the computer to execute the oxidation risk determination using the change rate of the oxidation power index value per unit sulfite or bisulfite in the fourth embodiment of the present invention.

FIG. 18 is a graph showing a relation between an elapsed time from contact with the sulfite or bisulfite and an oxidation-reduction potential according to Example 1 of the present invention.

FIG. 19 is a graph showing a relation between the elapsed time from contact with the sulfite or bisulfite and the oxidation-reduction potential according to Example 3-1 of the present invention.

FIG. 20 is a graph showing a relation between the sulfite or bisulfite concentration and the oxidation-reduction potential according to Example 3-2 of the present invention.

FIG. 21 is a graph showing a relation between the elapsed time from contact with the sulfite or bisulfite and the oxidation-reduction potential according to Example 4 of the present invention.

### DETAILED DESCRIPTION OF EMBODIMENTS OF THE INVENTION

In order to solve the above problems, the present invention has the following features. Hereinafter, the present invention will be described in detail with reference to the drawings, but contents of the present invention are not limited to the drawings.

The present invention relates to an oxidation risk evaluation method of a separation membrane, and in an embodiment, a deposit on a surface of the separation membrane is collected and brought into contact with a solution containing a sulfite or bisulfite, and an oxidation potential of the deposit is evaluated based on a generated oxidative substance. When the deposit is deposited on the surface of the separation membrane, the oxidative substance and an oxidation reaction promoting substance in feed water are captured and accumulated on the surface of the separation membrane. As a result, the substance is present at a higher concentration on the surface of the separation membrane than that in the feed water and concentrate water. Therefore, even if a content in the feed water is very small, it is possible to evaluate an oxidation risk by evaluating an oxidation potential of the deposit.

A catalytic reaction in the embodiment of the present invention refers to a reaction of the oxidative substance and the oxidation reaction promoting substance with the sulfite or bisulfite described above and in the background art.

Examples of the separation membrane in the embodiment of the present invention include a separation membrane provided in a water treatment plant.

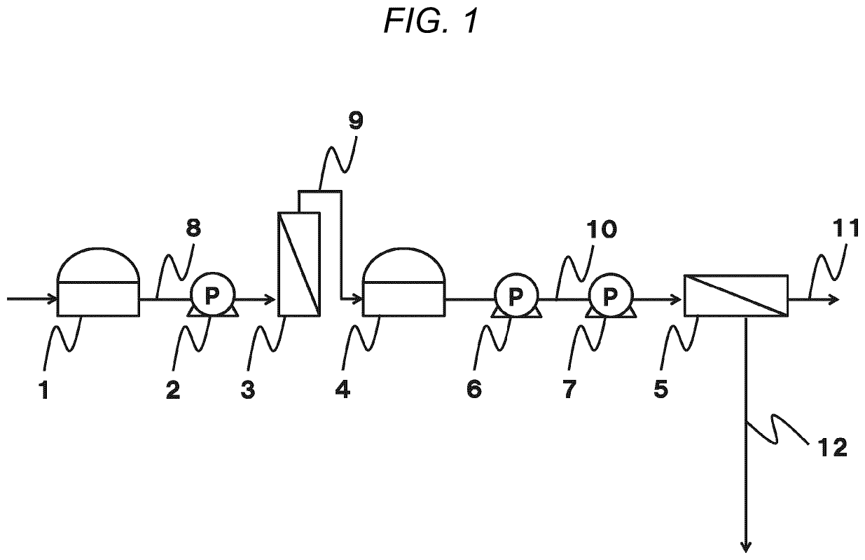

For example, FIG. 1 shows an example of a flow of a water treatment plant. The water treatment plant shown in FIG. 1 includes a raw water storage tank 1 for storing water to be treated (hereinafter, raw water), a raw water feed pump 2 for feeding the raw water, a pretreatment membrane filtration unit 3 for filtering the raw water, a pretreatment membrane filtrate water storage tank 4 for storing filtrate water of the pretreatment membrane filtration unit 3, a separation membrane filtration unit 5, a booster pump 6 for feeding the filtrate water of the pretreatment membrane filtration unit 3 to the separation membrane filtration unit 5, and a pressurizing pump 7 for pressurizing a pressure of the filtrate water of the pretreatment membrane filtration unit 3 to separate the filtrate water into permeate water and concentrate water by the separation membrane filtration unit 5.

The raw water storage tank 1 and the pretreatment membrane filtration unit 3 are connected by a raw water pipe 8, the pretreatment membrane filtration unit 3 and the pretreatment membrane filtrate water storage tank 4 are connected by a pretreatment membrane filtrate water pipe 9, and the pretreatment membrane filtrate water storage tank 4 and the separation membrane filtration unit 5 are connected by a separation membrane filtration feed water pipe 10. In the water treatment plant, the raw water is treated in the pretreatment membrane filtration unit 3, and pretreatment membrane filtrate water is temporarily stored in the pretreatment membrane filtrate water storage tank 4, then fed to the pressurizing pump 7 by the booster pump 6, pressurized by the pressurizing pump 7, fed to the separation membrane filtration unit 5, and separated into the permeate water from which a solute such as salt is removed and the concentrate water in which a solute such as salt is concentrated, and the permeate water and the concentrate water are discharged through a separation membrane filtration permeate water pipe 11 and a separation membrane filtration concentrate water pipe 12, respectively.

Examples of the separation membrane in the embodiment of the present invention include a separation membrane taken from a member installed in the separation membrane filtration unit, and a separation membrane in a member installed in an in-plant pipe such as the raw water pipe 8, the pretreatment filtrate water pipe 9, the separation membrane filtration feed water pipe 10, the separation membrane filtration permeate water pipe 11, and the separation membrane filtration concentrate water pipe 12.

The separation membrane in the embodiment of the present invention can be applied to a microfiltration membrane (MF), an ultrafiltration membrane (UF membrane), or the like as applied to the pretreatment filtration unit 3, but is particularly suitable for a plant in which separation and concentration of solute components are performed using a semipermeable membrane such as a nanofiltration membrane (NF membrane) or a reverse osmosis membrane (RO membrane), and is suitable for desalination of seawater or brackish water, production of industrial water, concentration of fruit juice or the like, advanced treatment in water supply, or the like. In addition, when the separation membrane of the present invention is a semipermeable membrane, the oxidation reaction promoting substance is concentrated and accumulated on a surface of the semipermeable membrane without permeation, and thus a concentration becomes higher than that in the raw water or the concentrate water, and an evaluation accuracy according to the present invention is improved, which is more desirable. These are usually provided in the separation membrane filtration unit 5.

The semipermeable membrane is a membrane having semipermeability that allows some components in the raw water, for example, a solvent to permeate therethrough and does not allow solute components such as salt to permeate therethrough, and examples thereof include a nanofiltration membrane (NF membrane) and a reverse osmosis membrane (RO membrane). Examples of a structure of the membrane include an asymmetric membrane having a dense separation layer on one surface of the membrane and having fine pores with a pore diameter gradually increasing from the dense separation layer toward the inside or the other surface of the membrane, and a composite membrane having a very thin separation functional layer made of another material on the dense layer of the asymmetric membrane. Examples of a membrane form include a hollow fiber membrane and a flat membrane.

The semipermeable membrane is generally used as an element in an appropriate form according to the membrane form. The semipermeable membrane element in the present invention is not particularly limited as long as the semipermeable membrane element has a substantial liquid chamber on both surface sides of the semipermeable membrane and is capable of pressurizing and permeating a liquid from one surface to the other surface of the semipermeable membrane. Examples include a spiral semipermeable membrane element having a semipermeable membrane in a flat membrane form. The spiral semipermeable membrane element generally includes a feed-side channel material that guides the feed water to the surface of the semipermeable membrane, the semipermeable membrane, and a permeate-side channel material that guides a liquid (permeate water) permeated through the semipermeable membrane to a water collecting pipe, and the feed-side channel material, the semipermeable membrane, and the permeate-side channel material are spirally wound around the water collecting pipe. As described above, the separation membrane of the present invention is preferably a separation membrane taken from a spiral semipermeable membrane element from a viewpoint of improving an evaluation accuracy.

In addition, by executing the evaluation method according to the embodiment of the present invention periodically, for example, regularly at a frequency of once a day to once a week, it is easy to identify an occurrence time of a sudden increase in the oxidation risk, and it is possible to quickly take measures. In this case, when it is difficult to take the separation membrane from an installation member of the separation membrane filtration unit, for example, a semipermeable membrane element, it is desirable to use a separation membrane installed in a pipe close to the separation membrane filtration unit, for example, the separation membrane filtration feed water pipe 10, the separation membrane filtration permeate water pipe 11, or the separation membrane filtration concentrate water pipe 12 because the oxidation risk in the separation membrane filtration unit 5 can be accurately and quickly evaluated. In particular, since the evaluation accuracy of the present invention is improved when a concentration of the oxidation promoting substance in the solution in the pipe is large, the separation membrane filtration feed water pipe or the separation filtration membrane concentrate water pipe is preferable, and the separation membrane filtration concentrate water pipe is more preferable. As an example of a mode of installing the separation membrane in the pipe, a water passing member including the separation membrane therein is installed in the pipe.

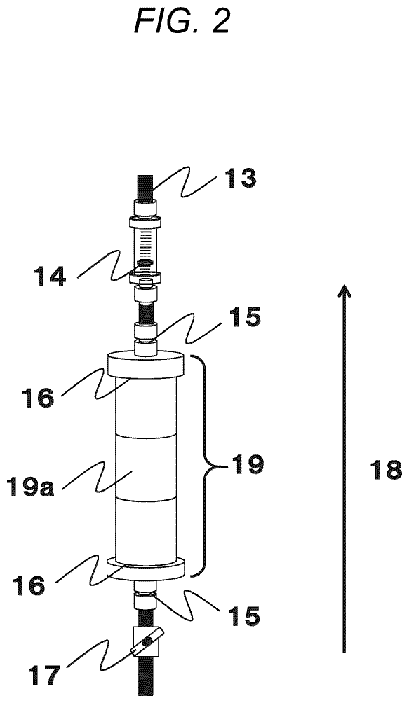

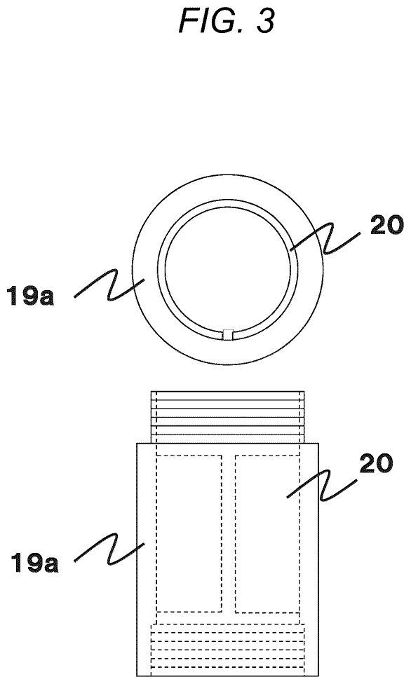

The water passing member in the embodiment of the present invention is not particularly limited as long as the water passing member includes a separation membrane therein and allows a solution to move from one end to the other end of the water passing member. In addition, as described above, in order to enable periodic and high-frequency oxidation risk evaluation, it is desirable that the water passing member be easily attached to and detached from a water treatment plant pipe. As an example, as shown in FIG. 2, it is desirable that a certain unit structure can be continuously coupled and separated via a connection member having a screw structure or a fitting structure (for example, a joint such as a one-touch joint 15), or it is desirable to have a structure in which a hose 13 can be easily cut using a scissor, or a structure that can be partially separated. As an example, as shown in FIG. 2 and FIG. 3, a water passing container opening and closing portion 16 and one or a plurality of cylindrical unit water passing members 19a each having a couplable structure in which screw grooves are provided at both ends in a longitudinal direction (water passing direction 18) of each unit water passing member 19a and a separation membrane 20 is installed are coupled, and the one or plurality of cylindrical unit water passing members 19a are further connected to a water passing container opening and closing portion 16 to form a water passing container 19, which is exemplified as a preferred mode. It is preferable that water leakage prevention measures such as a packing, a seal tape, and an O-ring be applied to a connection portion according to a shape. In this case, an area of the separation membrane 20 is not particularly limited, but is preferably 0.01 m² or more, and more preferably 0.03 m² or more. The separation membrane 20 is more preferably installed perpendicular to the water passing direction.

In addition, as a simple water passing container, a member such as a hose as described later may be used. In this case, the water passing container preferably has a cylindrical shape made of a soft material. Accordingly, the water passing container can be easily cut using a scissor or the like to take out the separation membrane inside. A flow meter 14 and a flow control valve 17 may be inserted in the middle of the hose 13.

When the water passing member is installed in the in-plant pipe other than the vicinity of the separation membrane filtration unit, it is also possible to evaluate the oxidation risk in each pipe and the vicinity.

A material (component) constituting the separation membrane or the semipermeable membrane is not particularly limited, but is preferably a cellulose acetate compound, a vinyl polymer compound, a polyamide compound, a polyester compound, a polyimide compound, or the like, and particularly preferably a cellulose acetate compound or a polyamide compound widely used as the semipermeable membrane material.

The sulfite or bisulfite is not particularly limited, and examples thereof include sodium sulfite, potassium sulfite, magnesium sulfite, calcium sulfite, sodium bisulfite, potassium bisulfite, magnesium bisulfite, and calcium bisulfite. These may use general commercial products, and among these, sodium sulfite, potassium sulfite, sodium bisulfite, and potassium bisulfite are particularly preferable.

In the embodiment of the present invention, the deposit on the surface of the separation membrane is collected by a physical method, and then subjected to the oxidation risk evaluation. A deposit collecting method is not particularly limited as long as the method is a quantitative method having a high collecting rate, and examples thereof include a method of immersing a membrane in pure water, and dispersing the deposit in pure water by ultrasonic grinding to collect the deposit. In addition, examples of a method of reliably peeling off and collecting the deposit adhered to the separation membrane include a method of collecting the deposit on the surface of the separation membrane using a wiping tool, and then immersing the wiping tool in pure water to disperse and collect the deposit in the pure water. Examples of the wiping tool include a cotton swab, a spatula, a scraper, and a rubber spatula, and it is desirable that these do not contain an oxidant-based disinfectant. In addition, in order to prevent breakage and peeling of the separation membrane, a cotton swab or a silicon tool is exemplified as a suitable tool, but is not particularly limited. As the pure water, distilled water, reverse osmosis membrane (RO membrane) water immediately after purification, ion-exchanged water, commercially available ultra-pure water, and the like are preferable. In addition, examples include a method of drying the separation membrane, and then collecting the deposit on the surface of the separation membrane in a container using the wiping tool described above. In this case, the separation membrane drying method is not particularly limited as long as the method is capable of easily peeling off the deposit on the surface of the separation membrane, and examples thereof include a method of performing natural drying at room temperature, or leaving the separation membrane to stand in a heated dryer and performing drying. The collected deposit is preferably dried at room temperature or dried by heating. In this case, it is desirable to perform drying by heating to 30° C. to 120° C. using an isothermal dryer until a weight change of the collected deposit becomes less than +0.1 g.

In the embodiment of the present invention, the deposit is brought into contact with the solution containing the sulfite or bisulfite, and an oxidation potential of the deposit is evaluated based on a generated oxidative substance. The method of bringing the deposit into contact with the solution containing the sulfite or bisulfite is not particularly limited, and examples thereof include a method of adding the collected deposit to a solution prepared by dissolving the sulfite or bisulfite in pure water. In this case, in order to increase a contact area between the deposit and the solution, it is desirable to use a stirring jig, a stirrer, a shaker, or the like to agitate and stir the solution in which the deposit is added. Examples of the stirring jig, the stirrer, and the shaker include a spoon, a spatula, a magnetic stirrer, a stirring rod having a stirring blade at a tip, a stirrer such as a three-one motor (manufactured by Shinto Scientific Co., Ltd.), and an ultrasonic cleaner.

In addition, another example of bringing the deposit into contact with the solution containing the sulfite or bisulfite to evaluate the oxidation potential of the deposit based on the generated oxidative substance is a method of using a deposit extracted liquid obtained by treating the deposit with a chemical solution. By using the deposit extracted liquid, a catalytic reaction between the oxidation reaction promoting substance in the deposit and the sulfite or bisulfite is promoted, and it is possible to more accurately evaluate the oxidation potential of the deposit. The method of treating and extracting the deposit with the chemical solution may use a general extraction operation. Examples include a method of adding the deposit to a chemical solution, leaving the chemical solution to stand or stirring the chemical solution using a stirring jig, a stirrer, or a shaker described later, and collecting the chemical solution after a certain time elapses. Examples of the method of collecting the chemical solution include a method of collecting the chemical solution using a jig such as a dropper, and a method of removing the deposit by a filtration operation to collect the chemical solution. As the chemical solution to be used, an acidic chemical solution is desirable. That is, a chemical solution having a hydrogen ion concentration of pH 6 or less is preferably used, and pH 3 or less is further preferably used, and examples thereof include sulfuric acid, nitric acid, hydrochloric acid, phosphoric acid, citric acid, oxalic acid, acetic acid, and formic acid, and among these, nitric acid, hydrochloric acid, and phosphoric acid are particularly preferable. These chemical solutions are generally commercially available and commercially available products can be used.

In addition, the solution containing the sulfite or bisulfite in the embodiment of the present invention may contain another inorganic salt in order to reproduce an actual state in the water treatment plant. In particular, sodium chloride is preferably contained. A sodium chloride concentration is preferably 500 mg/L to 80000 mg/L, and more preferably 1000 mg/L to 50000 mg/L.

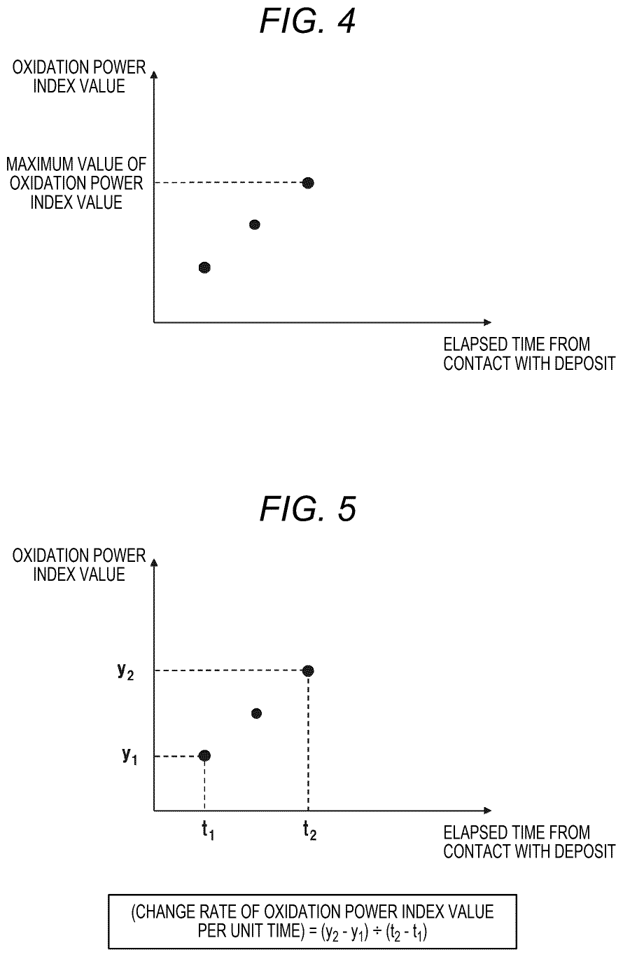

Examples of a first embodiment of evaluating the oxidation potential of the present invention include a method of bringing the deposit collected from the surface of the separation membrane into contact with the solution containing the sulfite or bisulfite, and measuring a change over time in an oxidation power index value of the solution. An example of the first embodiment is a method of evaluating at least any one of the following values as shown in FIG. 4 or FIG. 5. In this case, magnitude of the oxidation risk follows the determination method in the used oxidation power index value measurement method as described later, but in general, in any of the following values, it is determined that the larger the value, the larger the oxidation potential and the higher the oxidation risk.
A: maximum value of oxidation power index value
B: change rate of oxidation power index value per unit time
C: maximum value of moving average value of oxidation power index value
D: change rate of moving average value of oxidation power index value per unit time

The change rate of the oxidation power index value per unit time is calculated by dividing a difference in oxidation power index values by a difference in corresponding elapsed times from contact with the deposit. For example, in the case of FIG. 5, the change rate is calculated by Equation 1.

$$( change rate of oxidation power index value per unit time ) = ( {y}_{2} - {y}_{1} ) \div ( {t}_{2} - {t}_{1} ) \qquad ( Equation 1 )$$

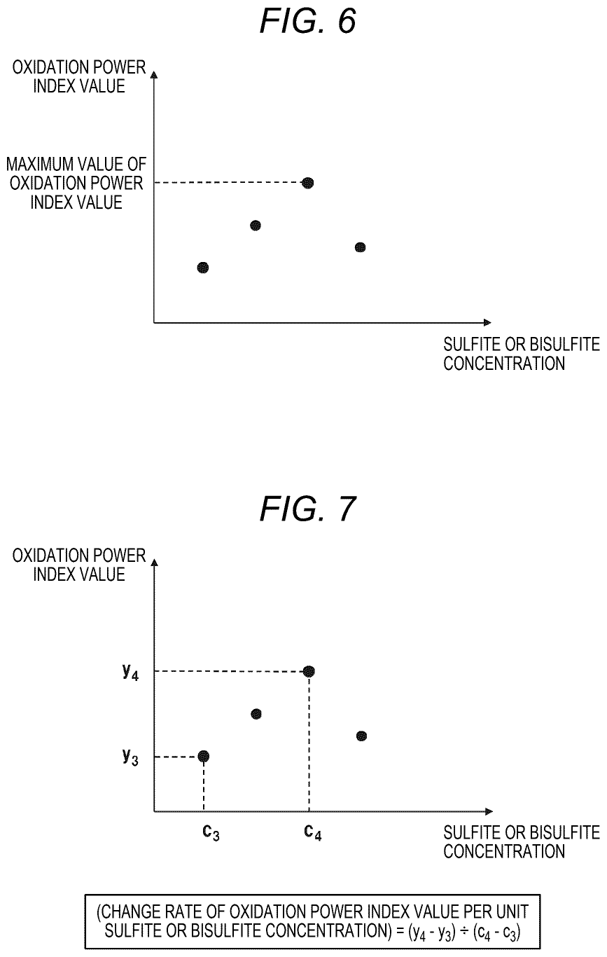

In Equation 1, y₁ represents an oxidation power index value when an elapsed time from the contact with the deposit is t₁, and y₂ represents an oxidation power index value when an elapsed time from the contact with the deposit is y₂. Here, t₂>t₁. When the calculation related to a plurality of t₂ and t₁ is performed in one evaluation in which the deposit is brought into contact once, a maximum value among values calculated by Equation 1 is adopted. A moving average value of the oxidation power index values refers to an arithmetic average of the oxidation power index values at the latest N (integer of 2 or more) points. Examples of a second embodiment of evaluating the oxidation potential of the present invention include a method of measuring oxidation power index values when two or more kinds of solutions having different sulfite or bisulfite concentrations are used and a deposit collected from the surface of the separation membrane is brought into contact with each of the solutions. An example of the second embodiment is a method of evaluating at least any one of the following values as shown in FIG. 6 or FIG. 7. In this case, magnitude of the oxidation risk follows the determination method in the used oxidation power index value measurement method as described later, but in general, in any of the following values, it is determined that the larger the value, the larger the oxidation potential and the higher the oxidation risk.
A: maximum value of oxidation power index value
B: change rate of oxidation power index value per unit sulfite or bisulfite concentration

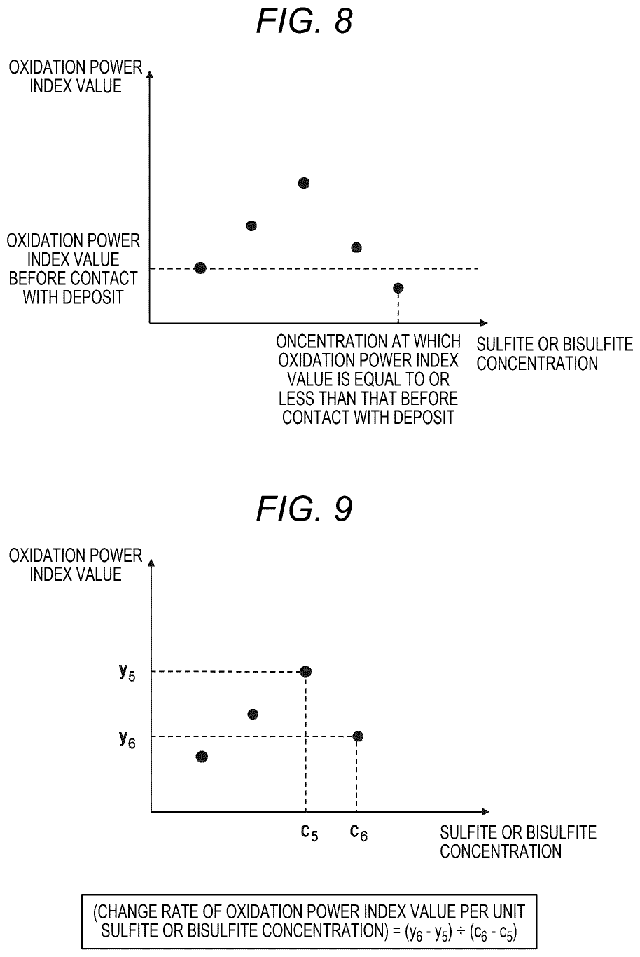

The change rate of the oxidation power index value per unit sulfite or bisulfite concentration is calculated by dividing a difference in the oxidation power index values by a difference in the corresponding sulfite or bisulfite concentrations. For example, in the case of FIG. 8, the change rate is calculated by Equation 2.

$$( change rate of oxidation power index value per unit sulfite or bisulfite concentration ) = ( {y}_{4} - {y}_{3} ) \div ( {c}_{4} - {c}_{3} ) \qquad ( Equation 2 )$$

In Equation 2, y₃ represents an oxidation power index value when the sulfite or bisulfite concentration is c₃, and y₄ represents an oxidation power index value when the sulfite or bisulfite concentration is c₄. Here, c₄>c₃.

When the calculation related to a plurality of c₄ and c₃ is performed in one evaluation in which the deposit is brought into contact once, a maximum value among values calculated by Equation 2 is adopted. When the oxidation power index values are measured over time under each of concentration conditions, any one of the following values is used as the oxidation power index value under the concentration condition.
A: maximum value of oxidation power index value
B: change rate of oxidation power index value per unit time
C: maximum value of moving average value of oxidation power index value
D: change rate of moving average value of oxidation power index value per unit time

Examples of a third embodiment of evaluating the oxidation potential of the present invention include a method of further adding a sulfite or bisulfite to the solution containing the sulfite or bisulfite which has been brought into contact with the deposit to change the sulfite or bisulfite concentration in the solution, and measuring the oxidation power index value when the deposit is brought into contact with the solution. An example of the third embodiment is a method of evaluating at least any one of the following values as shown in FIG. 6 or FIG. 7. In this case, magnitude of the oxidation risk follows the determination method in the used oxidation power index value measurement method as described later, but in general, in any of the following values, it is determined that the larger the value, the larger the oxidation potential and the higher the oxidation risk.
A: maximum value of oxidation power index value
B: change rate of oxidation power index value per unit sulfite or bisulfite concentration

The change rate of the oxidation power index value per unit sulfite or bisulfite concentration is calculated by dividing the difference in the oxidation power index values by the difference in the corresponding sulfite or bisulfite concentrations. For example, as described above, in the case of FIG. 7, the change rate is calculated by Equation 2.

Examples of a fourth embodiment of evaluating the oxidation potential of the present invention include a method of measuring the oxidation power index value when the deposit is brought into contact with a solution which contains the sulfite or bisulfite brought into contact with the deposit and has a concentration higher than that of the solution according to any one of the first to third embodiments. An example of the fourth embodiment is a method of evaluating at least any one of the following values as shown in FIG. 8 or FIG. 9. In this case, magnitude of the oxidation risk follows the determination method in the used oxidation power index value measurement method as described later, but in general, in any of the following values, it is determined that the larger the value, the larger the oxidation potential and the higher the oxidation risk.
A: sulfite or sulfite concentration at which oxidation power index value is equal to or less than that before contact with deposit
B: change rate of oxidation power index value per unit sulfite or bisulfite concentration

The change rate of the oxidation power index value per unit sulfite or bisulfite concentration is calculated by dividing the difference in the oxidation power index values by the difference in the corresponding sulfite or bisulfite concentrations. For example, in the case of FIG. 9, the change rate is calculated by Equation 3.

$$( change rate of oxidation power index value per unit sulfite or bisulfite concentration ) = ( {y}_{6} - {y}_{5} ) \div ( {c}_{6} - {c}_{5} ) \qquad ( Equation 3 )$$

In Equation 3, y₅ represents an oxidation power index value when the sulfite or bisulfite concentration is c₅, and y₆ represents an oxidation power index value when the sulfite or bisulfite concentration is c₆. Here, c₆>c₅. In general, since y₆<y₅, the value calculated by Equation 3 is negative, and the larger the absolute value, the smaller the value.

When the calculation related to a plurality of c₅ and c₆ is performed in one evaluation in which the deposit is brought into contact once, a maximum value among values calculated by Equation 3 is adopted.

When the oxidation power index values are measured over time under each of the concentration conditions, any one of the following values is used as the oxidation power index value under the concentration condition.
A: maximum value of oxidation power index value
B: change rate of oxidation power index value per unit time
C: maximum value of moving average value of oxidation power index value
D: change rate of moving average value of oxidation power index value per unit time

In addition, an example of a fifth embodiment of evaluating the oxidation potential of the present invention includes a method of measuring the oxidation power index value when the sulfite or bisulfite is further added after the oxidation power index value of the solution containing the sulfite or bisulfite which has been brought into contact with the deposit according to any one of the first to third embodiments becomes maximum. An example of the fifth embodiment is a method of evaluating at least any one of the following values as shown in FIG. 8 or FIG. 9. In this case, the magnitude of the oxidation risk follows the determination method in the used oxidation power index value measurement method as described later, but in general, in any of the following values, it is determined that the larger the value, the larger the oxidation potential and the higher the oxidation risk.
A: sulfite or sulfite concentration at which oxidation power index value is equal to or less than that before contact with deposit
B: change rate of oxidation power index value per unit sulfite or bisulfite concentration

The change rate of the oxidation power index value per unit sulfite or bisulfite concentration is calculated by dividing the difference in the oxidation power index values by the difference in the corresponding sulfite or bisulfite concentrations, and is generally a negative value. For example, as described above, in the case of FIG. 9, the change rate is calculated by Equation 3.

By using any one of the first to third embodiments, it is possible to quickly and easily evaluate the oxidation potential. For example, in a case of evaluating the oxidation risk at a specified concentration amount such as a sulfite or bisulfite concentration used in an actual plant, the first embodiment is preferable. On the other hand, when it is desired to know the maximum oxidation risk that is concerned, the second or third embodiment is preferable. In addition, when the fourth or fifth embodiment is used, the sulfite or bisulfite concentration necessary to sufficiently counteract the oxidation potential can be known.

Further, in the first to fifth embodiments, when a membrane area used for collecting the deposit and the number of years of use (operation) of the element or the water passing member containing the membrane can be known, the oxidation risk in the actual plant can be more accurately evaluated by using a value obtained by dividing an index obtained in each of the embodiments by the membrane area and/or the number of years of use. For example, in the case of using the first embodiment, it is possible to more accurately evaluate the oxidation risk by using a value calculated by Equation 4.

$$( oxidation power index value per unit area \cdot number of years of use ) = z \div ( ( membrane area for collecting deposit ) \times ( number of years of use of membrane ) ) \qquad ( Equation 4 )$$

Here, z represents the oxidation risk index value evaluated in the first embodiment, that is, any one of the following values.
A: maximum value of oxidation power index value
B: change rate of oxidation power index value per unit time
C: maximum value of moving average value of oxidation power index value
D: change rate of moving average value of oxidation power index value per unit time

In other embodiments, the oxidation risk index value evaluated by methods is also set to z, and the value calculated by Equation 4 is used, so that it is possible to more accurately evaluate the oxidation risk.

The oxidation power index value measurement method is not particularly limited, but a method of sensitively reacting with an oxidative substance is desirable. Examples of a general method include an oxidation-reduction potential (ORP) measurement, a measurement of a free residual chlorine concentration or a combined and/or total chlorine concentration performed by N, N-diethyl para phenylenediamine (hereinafter, referred to as DPD) method, a Fujiwara test described in Patent Literature 1 and Non Patent Literature 1, and a dissolved oxygen amount (DO) measurement. In particular, from a viewpoint of ease of measurement, the ORP measurement, the measurement of the free residual chlorine concentration and the combined and/or total chlorine concentration performed by the DPD method are preferable.

An oxidation-reduction potential (ORP) is an index of oxidizability or reducibility of a solution, is determined by equilibrium in electron donation/acceptance between oxidant and reductant coexisting in the solution, and is generally measured as a potential difference between a metal electrode and a reference electrode based on a Nernst equation. A method of measuring the index includes a method using an oxidation-reduction potential meter, and the oxidation-reduction potential meter is not particularly limited, but is preferably an oxidation-reduction potential meter that measures an oxidation-reduction potential based on a Nernst equation using a platinum electrode and a reference electrode or a composite electrode of a platinum electrode and a reference electrode, and preferably, the measurement is performed using a saturated calomel electrode, a saturated silver/silver-chloride electrode, or the like as the reference electrode, and more preferably using a 3.3 mol/L silver chloride electrode as the reference electrode.

The DPD method is a method of measuring the free residual chlorine concentration or the combined and/or total chlorine concentration based on a color reaction between an oxidative substance present in water and a DPD reagent. When the index is measured, a commercially available measuring instrument and DPD reagent can be used.

When an index affected by a measurement condition is used as the oxidation power index value, it is desirable to use a value under any one condition or a value obtained by uniquely correcting an influence of the measurement condition. For example, when the oxidation-reduction potential (ORP) is used as the index described above, it is desirable to use a value under particular measurement conditions, particularly under particular solution temperature and pH, and it is preferable to use a value under particular one condition of a solution temperature of 20° C. to 35° C. and pH of 1 to 8. Hereinafter, unless the oxidation-reduction potential is otherwise specified, a value at a solution temperature of 25° C. and a pH of 7 is described.

As an example of the oxidation risk determination method, when the oxidation potential is evaluated by the maximum value of the oxidation power index value or the maximum value of the moving average value of the oxidation power index values among the first to third embodiments, for example, when the oxidation-reduction potential is used as the oxidation power index value, it can be determined that the oxidation risk is low (mild) if the maximum value is less than 300 mV, the oxidation risk is present (moderate) if the maximum value is 300 mV or more and less than 600 mV, and the oxidation risk is high (severe) if the maximum value is 600 mV or more. When the free residual chlorine concentration or the total chlorine concentration is used as the oxidation power index value, it can be determined that the oxidation risk is low (mild) if the concentration is less than 0.01 mg/L, the oxidation risk is present (moderate) if the concentration is 0.01 mg/L or more and less than 0.5 mg/L, and the oxidation risk is high (severe) if the concentration is 0.5 mg/L or more.

In addition, as an example of the oxidation risk determination method, when the oxidation potential is evaluated by the change rate of the oxidation power index value per unit time or the change rate of the moving average value of the oxidation power index value per unit time in the first embodiment, for example, when the oxidation-reduction potential is used as the oxidation power index value, it can be determined that the oxidation risk is low (mild) if the change rate is less than 50 mV/min, the oxidation risk is present (moderate) if the change rate is 50 mV/min or more and less than 300 mV/min, and the oxidation risk is high (severe) if the change rate is 300 mV/min or more. When the free residual chlorine concentration or the total chlorine concentration is used as the oxidation power index value, it can be determined that the oxidation risk is low (mild) if the change rate is 0.1 mg/L/min, the oxidation risk is present (moderate) if the change rate is 0.1 mg/L/min or more and less than 0.5 mg/L/min, and the oxidation risk is high (severe) if the change rate is 0.5 mg/L/min or more.

In addition, when the oxidation potential is evaluated by the change rate of the oxidation power index value per unit concentration in the second or third embodiment, for example, when the oxidation-reduction potential is used as the oxidation power index value, it can be determined that the oxidation risk is low (mild) if the change rate is less than 50 mV/(mg/L), the oxidation risk is present (moderate) if the change rate is 50 mV/(mg/L) or more and less than 100 mV/(mg/L), and the oxidation risk is high (severe) if the change rate is 100 mV/(mg/L) or more. When the free residual chlorine concentration or the total chlorine concentration is used as the oxidation power index value, it can be determined that the oxidation risk is low (mild) if the change rate is 0.1 mg/L/(mg/L), the oxidation risk is medium (moderate) if the change rate is 0.1 mg/L/(mg/L) or more and less than 0.5 mg/L/(mg/L), and the oxidation risk is high (severe) if the change rate is 0.5 mg/L/(mg/L) or more.

In addition, in the fourth or fifth embodiment, when the oxidation potential is evaluated by the sulfite or bisulfite concentration at which the oxidation power index value is equal to or less than that before contact with the deposit, it can be determined that the oxidation risk is low (mild) if the concentration is less than 5 mg/L, the oxidation risk is present (moderate) if the concentration is 5 mg/L or more and less than 100 mg/L, and the oxidation risk is high (severe) if the concentration is 100 mg/L or more. In addition, when the oxidation potential is evaluated by the change rate of the oxidation power index value per unit sulfite or bisulfite concentration, for example, when the oxidation-reduction potential is used as the oxidation power index value, it can be determined that the oxidation risk is low (mild) if the change rate is less than −100 mV/(mg/L), the oxidation risk is present (moderate) if the change rate is −100 mV/(mg/L) or more and less than −5 mV/(mg/L), and the oxidation risk is high (severe) if the change rate is −5 mV/(mg/L) or more. When the free residual chlorine concentration or the total chlorine concentration is used as the oxidation power index value, it can be determined that the oxidation risk is low (mild) if the change rate is less than −0.01 mg/L/(mg/L), the oxidation risk is present (moderate) if the change rate is −0.01 mg/L/(mg/L) or more and less than −5.0×10⁻³ mg/L/(mg/L), and the oxidation risk is high (severe) if the change rate is −5.0×10⁻³ mg/L/(mg/L) or more.

When the deposit is brought into contact with the solution containing the sulfite or bisulfite, if a hydrogen ion concentration index (pH) of the solution to be brought into contact is 9.0 or more, the generation of oxidative substances is promoted. Accordingly, since it is possible to quickly evaluate an oxidation power potential of the deposit with high accuracy, it is preferable to bring the deposit into contact with the solution which contains the sulfite or bisulfite having a hydrogen ion concentration index (pH) of 9.0 or more.

Examples of a method of adjusting the hydrogen ion concentration index (pH) of the solution containing the sulfite or bisulfite to 9.0 or more include a method of adding a basic substance to the solution. The basic substance is not particularly limited, and examples thereof include sodium hydroxide, potassium hydroxide, calcium hydroxide, magnesium hydroxide, sodium carbonate, potassium carbonate, sodium hydrogen carbonate, and potassium hydrogen carbonate.

The solution containing the sulfite or bisulfite according to the embodiment of the present invention preferably contains a chloride ion and/or a bromide ion. When these ions are present, oxidative substances such as hypochlorite ions and hypobromite ions are generated, and the oxidation risk increases, and thus the oxidation potential in the water treatment plant can be more accurately estimated. In addition, by making the solution containing the sulfite or bisulfite have composition close to raw water or feed water used in an actual water treatment plant, it is possible to more accurately evaluate the oxidation risk in the water treatment plant.

As a use example of the present invention, the oxidation risk in a certain water treatment plant is determined based on the oxidation potential evaluated in any one of the first to fifth embodiments of the present invention. In this case, the magnitude of the oxidation risk follows the determination method in the used oxidation power index value measurement method. For example, in the ORP or DPD method, the higher the measured value, the higher the oxidation risk, and in the Fujiwara test described in Patent Literature 1 or Non Patent Literature 1, it is determined that the oxidation risk is high when coloration occurs. Further, as a use example of the present invention, it is possible to determine oxidation risks at different water treatment plants or pipe locations, and at different times by comparing oxidation potentials evaluated using a certain specified amount of deposits by the same method of any one of the first to fifth embodiments of the present invention. For example, in a case where deposits are collected from a semipermeable membrane element or a water passing member having a separation membrane used in different plants, and the oxidation potentials of the respective deposits are evaluated using the same amount of the deposits by the same method of any one of the first to fifth embodiments, it is possible to compare the magnitude of the oxidation risks in the respective plants from the magnitude of the oxidation potentials. In addition, for example, it is possible to determine the magnitude of the oxidation risk in each of the pipes by installing water passing members having separation membranes in different pipes in the same plant and evaluating the oxidation potentials using the same amount of the deposits by the same method of any one of the first to fifth embodiments for the deposits collected from the separation membranes. Accordingly, for example, when the oxidation risk in the plant increases, it is easy to specify a cause location. In addition, by periodically evaluating the oxidation potential of the deposit in the same plant in any one of the first to fifth embodiments of the present invention, the oxidation risk of the plant can be monitored, and even when the oxidation risk increases, it is possible to quickly cope with the increase. For example, it is possible to detect an increase in the oxidation risk due to excessive addition of a reducing agent in the feed water line or due to a trouble in a pre-process. In addition, it is possible to find presence or absence of an oxidation risk in a permeate water line due to deterioration of the semipermeable membrane, damage to members, or the like (trouble of mixing of the oxidation reaction promoting substance into the permeate water). As described above, according to the present invention, it is possible to perform simple and quick oxidation risk determination, which is difficult in the method in the related art, and the present invention is useful for risk avoidance and early solution of operation trouble.

Examples of another embodiment of the present invention include an oxidation risk evaluation program for a water treatment plant, in the oxidation risk evaluation program, in order for a computer to evaluate an oxidation risk, a data recording unit that records input data in the computer, and a unit that evaluates an oxidation potential of a deposit based on the input data are included, and the input data includes a condition and an oxidation power index value when the deposit collected from the used separation membrane is brought into contact with a solution containing a sulfite or bisulfite. The present embodiment causes the computer having each units to function to evaluate the oxidation risk of the separation membrane. The present embodiment can be recorded in a recording device such as a memory or a hard disk of the computer, and a form of recording is not particularly limited. Examples of another embodiment of the present invention include a recording medium storing the evaluation risk evaluation program.

The computer includes the data recording unit that records the condition and the oxidation power index value when the deposit collected from the used separation membrane is brought into contact with the solution containing the sulfite or bisulfite. Further, the oxidation risk index value is determined or calculated using the data recorded in the data recording unit, and is output. Alternatively, the oxidation risk of the separation membrane is evaluated and output based on a predetermined determination criterion.

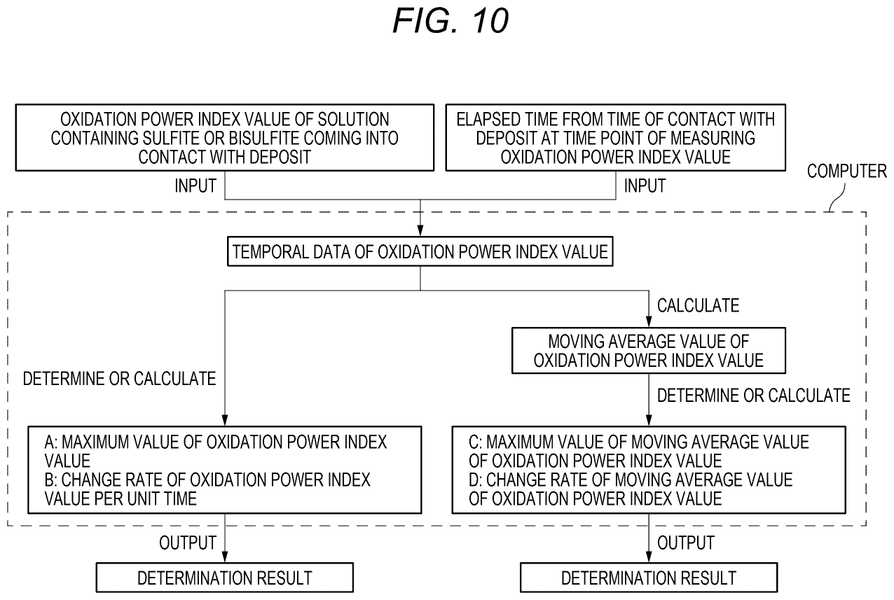

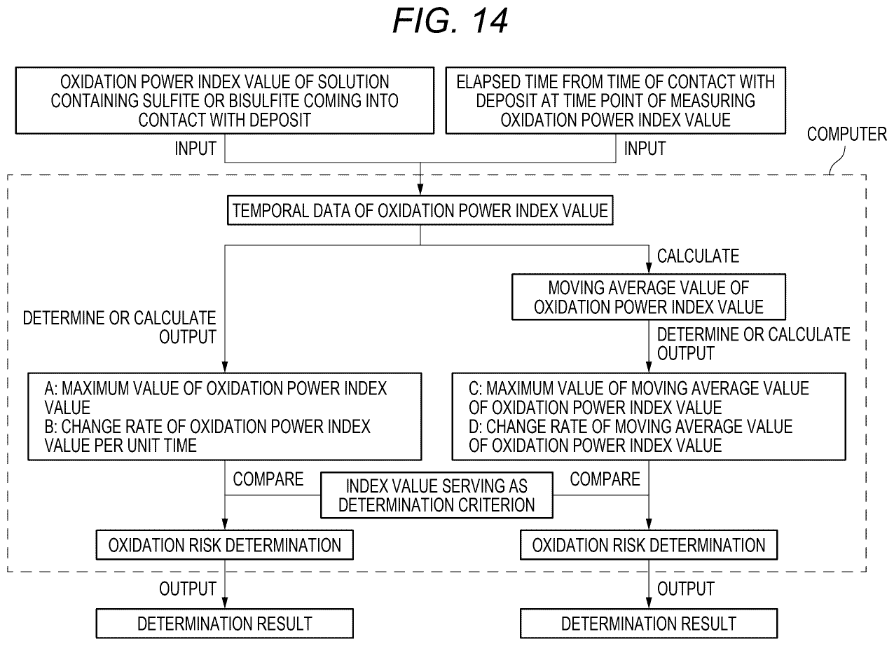

For example, as shown in FIG. 10 or FIG. 14, a program that evaluates the oxidation risk based on data over time of the oxidation power index value is exemplified. That is, any one of the following values is determined or calculated based on the input oxidation power index value of the solution containing the sulfite or bisulfite brought into contact with the deposit and the input elapsed time from the time of contact with the deposit at a time point of measuring the oxidation power index value, so that the oxidation potential is evaluated, and the oxidation risk determination result is output.
A: maximum value of oxidation power index value
B: change rate of oxidation power index value per unit time
C: maximum value of moving average value of oxidation power index value
D: change rate of moving average value of oxidation power index value per unit time

In this case, the output determination result is preferably the oxidation potential evaluation result itself which is the oxidation risk index value as shown in FIG. 10 in order to accurately know the oxidation risk. On the other hand, in order to simplify and facilitate discrimination, a result is preferable one obtained by determining the magnitude of the oxidation risk, for example, a degree of oxidation degradation (mild, moderate, severe) based on the predetermined determination criterion (index value) as shown in FIG. 14. As the index value and the determination criterion in this case, as described above, for example, a value and a determination criterion in line with the oxidation potential evaluation method can be used, such as the oxidation-reduction potential, the free residual chlorine concentration, or the combined (total) chlorine concentration can be used.

In the oxidation potential evaluation method, when it is desired to quickly perform the determination, it is preferable to use the change rate of the oxidation power index value per unit time (B). On the other hand, from a viewpoint of accuracy of determination, the maximum value of the oxidation power index value (A) is preferable. For example, when it is desired to accurately know the oxidation risk in a certain period of time, a moving average value is calculated based on the recorded data over time, and the oxidation potential is evaluated by the maximum value of the moving average value (C) or the change rate of the moving average value (D), so that the oxidation risk can be determined. In this case, it is preferable to use the change rate of the moving average value (D) when it is desired to quickly obtain the result, and it is preferable to use the maximum value of the moving average value (C) when it is desired to accurately know the oxidation risk.

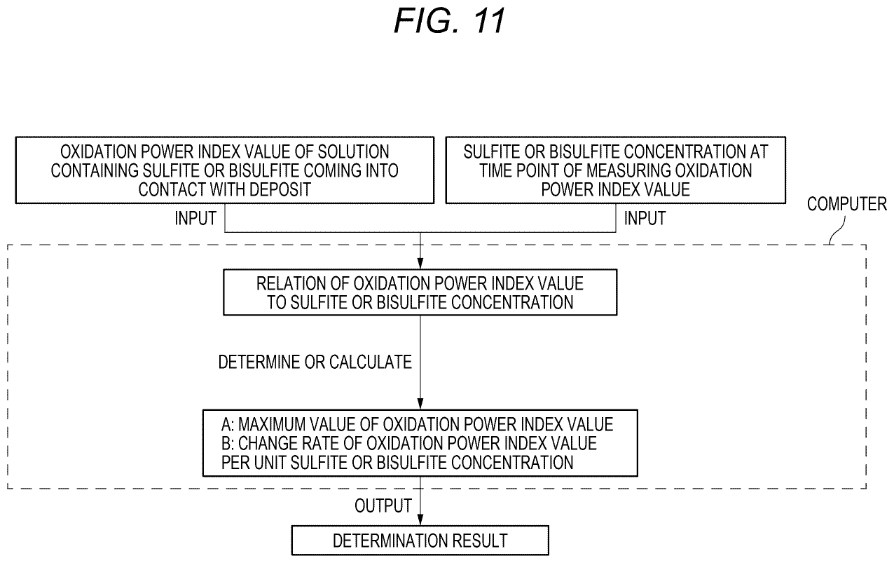

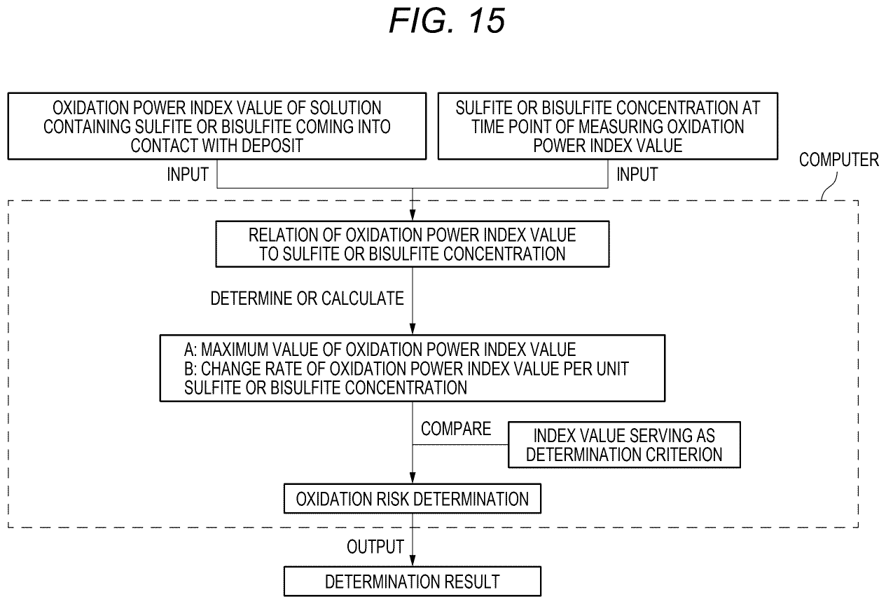

In addition, for example, as shown in FIG. 11 or FIG. 15, a program that evaluates the oxidation risk based on a relation of the oxidation power index value to the sulfite or bisulfite concentration is exemplified. That is, any one of the following values is determined or calculated based on the oxidation power index value of the solution containing the sulfite or bisulfite brought into contact with the deposit and the sulfite or bisulfite concentration at the time point of measuring the oxidation power index value, so that the oxidation potential is evaluated, and the oxidation risk determination result is output.
A: maximum value of oxidation power index value
B: change rate of oxidation power index value per unit sulfite or bisulfite concentration

In this case, the output determination result is preferably the value of the oxidation potential evaluation result itself representing the magnitude of the oxidation risk as shown in FIG. 11 in order to accurately know the oxidation risk. On the other hand, in order to simplify and facilitate discrimination, a result is preferable one obtained by performing determination based on the predetermined determination criterion (index value) as shown in FIG. 15.

As the index value in this case, as described above, a value or a determination criterion in line with the oxidation potential evaluation method can be used.

In addition, as described above, when it is desired to quickly perform the determination, the change rate of the oxidation power index value per unit sulfite or bisulfite (B) can be used. On the other hand, from the viewpoint of accuracy of determination, the maximum value of the oxidation power index value (A) is preferable.

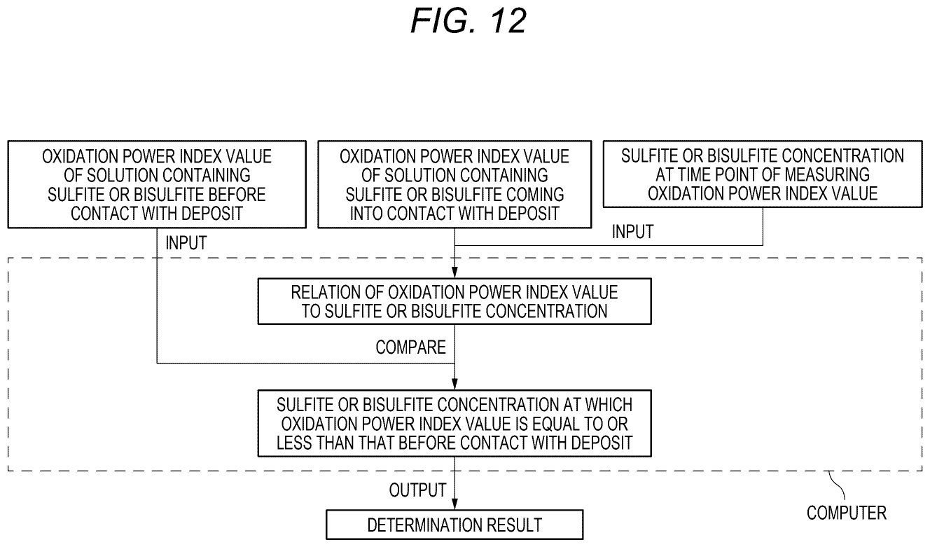

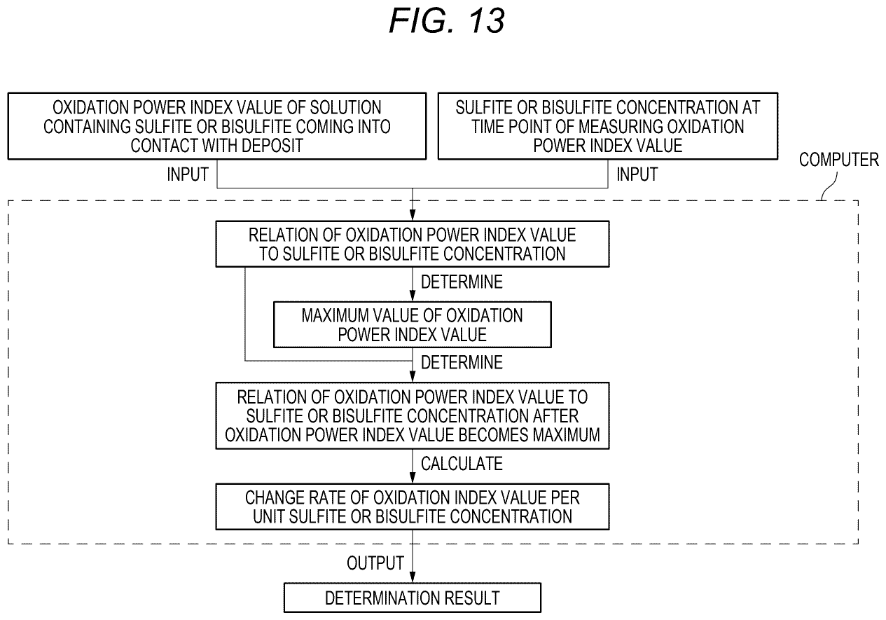

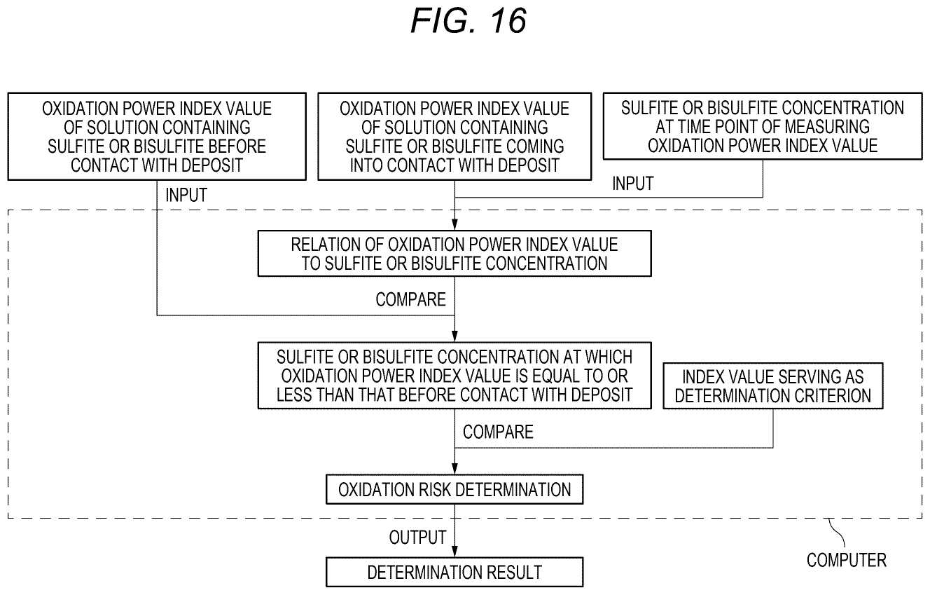

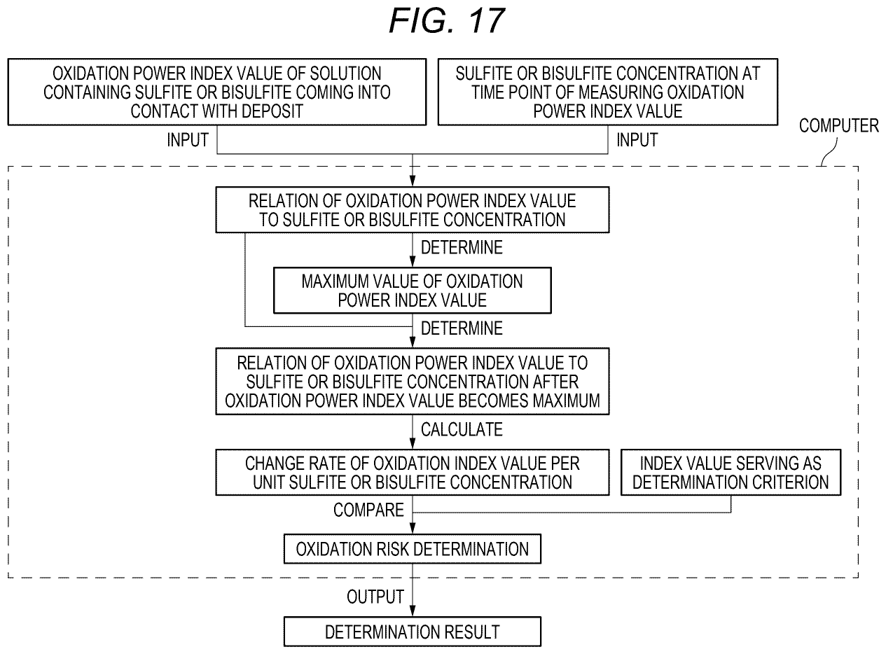

As another example, there is a program that evaluates the oxidation risk based on the relation of the oxidation power index value to the sulfite or bisulfite concentration after the oxidation power index value becomes maximum. That is, for example, as shown in FIG. 12 or 16, there is a program that determines the oxidation risk by the sulfite or bisulfite concentration at which the oxidation power index value is equal to or less than that before contact with the deposit, based on the oxidation power index value of the solution containing the sulfite or bisulfite brought into contact with the deposit, the sulfite or bisulfite concentration at the time point of measuring the oxidation power index value, and the oxidation power index value of the solution containing the sulfite or bisulfite before contact with the deposit. As another example, as shown in FIG. 13 or 17, there is a program in which the change rate of the sulfite or bisulfite concentration is calculated based on the relation of the oxidation power index value to the sulfite or bisulfite concentration after the oxidation power index value becomes maximum from the oxidation power index value of the solution containing sulfite or bisulfite brought into contact with the deposit and the sulfite or bisulfite concentration at the time point of measuring the oxidation power index value, so that the oxidation potential is evaluated, and the oxidation risk is determined.

In this case, the output determination result is preferably the value of the oxidation potential evaluation result itself representing the magnitude of the oxidation risk as shown in FIG. 12 or 13 in order to accurately know the oxidation risk. On the other hand, in order to simplify and facilitate discrimination, a result is preferable one obtained by determining the magnitude of the oxidation risk based on the index value serving as the predetermined determination criterion as shown in FIG. 16 or 17.

As described above, which of the programs using the first to fifth embodiments is to be used can be freely selected according to the situation or purpose.

An example of another embodiment of the present invention is an oxidation risk evaluation device for a water treatment plant, the oxidation risk evaluation device including a data input unit that inputs, to a computer, a condition and an oxidation power index value when a deposit collected from a used separation membrane is brought into contact with a solution containing a sulfite or bisulfite, a data recording unit that records the condition and the oxidation power index value in the computer, and a unit that evaluates an oxidation potential by the method described above. The evaluation device includes the data input unit configured to input the condition and the oxidation power index value when the deposit collected from the separation membrane is brought into contact with the solution containing the sulfite or bisulfite, the data recording unit configured to record the input data in the computer, the data recording unit configured to record the input data in the computer, and a method of evaluating the oxidation potential of the deposit based on the input data.

Examples of the condition when the deposit is brought into contact include an elapsed time from contact, the sulfite or bisulfite concentration, and an oxidation power index value before the contact with deposit. Examples of the data input unit include a method in which the conditions and the oxidation power index value when the deposit collected from the used separation membrane is brought into contact with the solution containing the sulfite or bisulfite are input based on the evaluation results, and these can be input by manually inputting numerical values or by automatically inputting these by using the evaluation device itself equipped with a method of evaluating and measuring these conditions. As the automatic input method, for example, an evaluation and measurement method capable of determining the elapsed time from the contact and the sulfite or bisulfite concentration is provided in the evaluation device, and the result is recorded and input at any time. Examples of such an evaluation and measurement method include a method of determining the sulfite or bisulfite concentration based on a calibration curve created by using a colorimetric method using a chemical agent that reacts with the sulfite or bisulfite to be colored or change color, and further determining the elapsed time from the contact based on time.

As the method of evaluating the oxidation potential, the present invention, that is, the method of performing the evaluation based on the data over time of the oxidation power index value and the relation of the oxidation power index value to the sulfite or bisulfite concentration as described above and as in FIG. 10 to FIG. 17 is exemplified, and the determined and calculated oxidation risk index value is output. Alternatively, the oxidation risk of the separation membrane is evaluated based on the predetermined determination criterion and output.

### EXAMPLES

Hereinafter, the present invention will be described in more detail using Examples, but the present invention is not limited to these Examples.

### Example 1

In a water treatment plant A operated for about 2 months, a water quality of product water tended to deteriorate. A semipermeable membrane element TM820C-400 (manufactured by Toray Industries, Inc.) used in the plant was disassembled to take a separation membrane. The separation membrane was dried at room temperature for 24 hours, and a deposit was collected using a silicon jig and was added to a glass petri dish. The deposit was dried in a dryer set at 120° C. for 2 hours. The product is referred to as a deposit A-1.

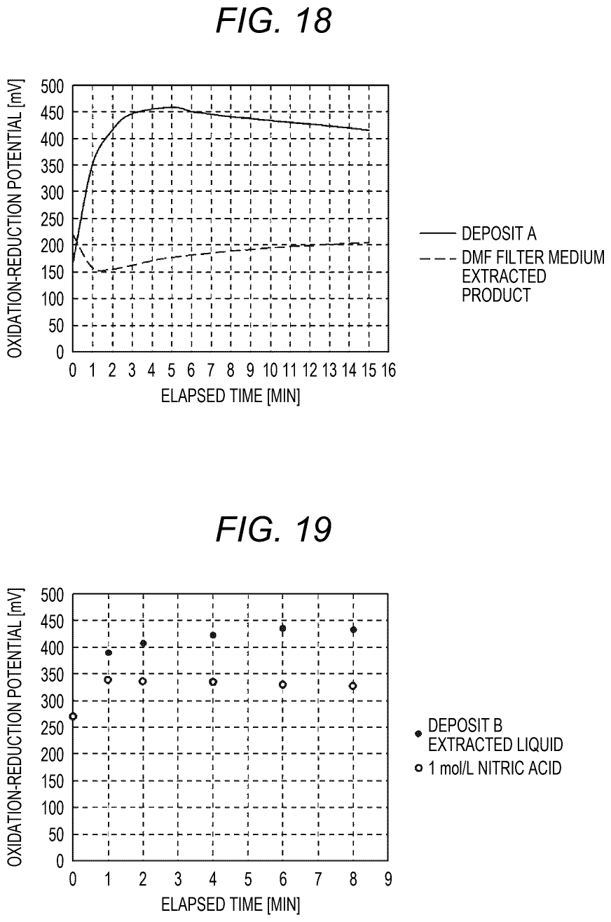

In order to prepare 400 mL of an aqueous solution having a sodium bisulfite concentration of 20 mg/L in a 1 L beaker, seawater, sodium bisulfite, and a stirrer bar were put, followed by stirring with a magnetic stirrer to dissolve these. Further, a 1 mol/L sodium hydroxide aqueous solution and 1 mol/L sulfuric acid were added to adjust pH to 7. To this solution, 10 mg of the above deposit A-1 was added, and a change over time in oxidation-reduction potential was measured using an oxidation-reduction potential meter while stirring. The pH and a solution temperature were measured using a hydrogen ion concentration (pH) meter and an alcohol thermometer. The results are shown in FIG. 18. The maximum value of the oxidation-reduction potential was 459 mV. In this case, the pH was 7 and the solution temperature was 25° C. Since the oxidation-reduction potential is 300 mV or more, the oxidation potential of the deposit is high, and the oxidation risk in the present plant is considered to be high.

In order to examine presence or absence of oxidation degradation of the separation membrane, 6 g of pyridine and 2 g of a 3 mol/L sodium hydroxide aqueous solution were added into a 20 mL glass bottle and mixed. The separation membrane taken from the disassembled element was cut into a 10 cm square and rinsed with pure water to peel and remove a substrate. When the obtained product was added into the pyridine-sodium hydroxide aqueous solution described above and allowed to stand at room temperature for 8 hours, the solution turned red in color. From this result, the oxidation degradation is considered to be occurred in the separation membrane as described in Non Patent Literature 1. In addition, as a result of examining elemental composition of the deposit described above by high-frequency inductively coupled plasma (ICP) emission analysis, it was found that copper (1.1 wt %) and manganese (32 wt %) were contained.

### Comparative Example 1

A filter medium (sand) of a gravity filter (DMF) used in the water treatment plant A at the same time as in Example 1 was immersed in concentrated nitric acid for 20 hours to obtain a DMF filter medium extracted liquid. In order to prepare 400 mL of an aqueous solution having a sodium bisulfite concentration of 20 mg/L in a 1 L beaker, seawater, sodium bisulfite, and a stirrer bar were put, followed by stirring with a magnetic stirrer to dissolve these. To this solution, 5 g of the DMF filter medium extracted liquid described above was added, and the change over time in the oxidation-reduction potential was measured while stirring. The results are also shown in FIG. 18. The maximum value of the oxidation-reduction potential was 220 mV.

From the results of Example 1 and Comparative Example 1, the oxidation degradation occurs in the separation membrane element used in the water treatment plant A, and the oxidation risk is considered to be high in the water treatment plant A. The high oxidation risk in the water treatment plant A can be quickly determined by evaluating the oxidation potential using the deposit on the used separation membrane as described above. Further, since a large amount of copper and manganese was detected in the elemental composition analysis result of the deposit, it is presumed that the oxidation risk of the separation membrane is high as described above. On the other hand, when evaluation is performed using the DMF filter medium extracted product, the oxidation potential is low, the oxidation risk of the water treatment plant A cannot be correctly evaluated, and it is possible to evaluate the oxidation risk by using the deposit on the separation membrane.

### Example 2

The separation membrane was taken from the same semipermeable membrane element TM820C-400 (manufactured by Toray Industries, Inc.) as in Example 1. The separation membrane was dried at room temperature for 24 hours, and a deposit was collected using a silicon jig and was added to a glass petri dish. The deposit was dried in a dryer set at 120° C. for 2 hours. The product is referred to as a deposit A-2.

In order to prepare 400 mL of an aqueous solution having a sodium bisulfite concentration of 20 mg/L in a 1 L beaker, seawater, sodium bisulfite, and a stirrer bar were put, followed by stirring with a magnetic stirrer to dissolve these. Further, a 1 mol/L sodium hydroxide aqueous solution and 1 mol/L sulfuric acid were added to adjust pH to 7. To this solution, 10 mg of the deposit A-2 was added, and stirred for 15 minutes. A free chlorine concentration in this solution was measured using a residual chlorine meter and was 4.34 mg/L.

Since the free chlorine concentration was 0.5 mg/L or more, it was determined that the oxidation potential of the deposit was high and the oxidation risk in the present plant was high, similar to Example 1.

### Example 3-1

Regarding a water treatment plant B operated for about 0.5 months, the oxidation risk was examined using an oxidation risk evaluation method performed by a catalytic reaction in order to confirm a plant operation situation. A semipermeable membrane element TML10D (manufactured by Toray Industries, Inc.) used in the plant was disassembled to take a separation membrane. A deposit was collected using a silicon jig from the separation membrane, and was added in a glass petri dish. The deposit was dried in a dryer set at 120° C. for 2 hours. The product is referred to as a deposit B.

To a sealable glass bottle, 20 mL of a 1 mol/L nitric acid aqueous solution was added, and 1.0 g of the deposit B was further added, followed by sealing and standing at room temperature. After one night, the supernatant was collected in another glass bottle using a dropper. The product is referred to as a deposit extracted liquid B.

In order to prepare 500 mL of a sodium chloride aqueous solution having a sodium bisulfite concentration of 10 mg/L and a sodium chloride concentration of 32000 mg/L, sodium chloride, sodium bisulfite, pure water, and a stirrer bar were added in a 1 L beaker, followed by stirring with a magnetic stirrer to dissolve these. While stirring the solution, 5 mL of the deposit extracted liquid was added, and a change over time in the oxidation-reduction potential was measured for eight minutes using an oxidation-reduction potential meter. In this case, a pH and a temperature of the solution were also measured using a hydrogen ion concentration (pH) meter and an alcohol thermometer. A maximum value of the oxidation-reduction potential in eight minutes was 436 mV.

On the other hand, 5 mL of 1 mol/L nitric acid was added to the sodium chloride aqueous solution having the sodium bisulfite concentration of 10 mg/L and the sodium chloride concentration of 32000 mg/L, and the oxidation-reduction potential, the pH, and the temperature were measured, and the result was 338 mV at pH of 2 and 26° C.

Results of the above are shown in FIG. 19. In any case, the hydrogen ion concentration (pH) of the sodium bisulfite aqueous solution was 2 and the solution temperature was 26° C. during the measurement of the change over time.

From these results, since the oxidation potential of the deposit extracted liquid B was higher by 98 mV than that in the case where only 1 mol/L nitric acid was added to the sodium bisulfite aqueous solution without using the deposit, it was determined that the oxidation potential of the deposit B was high and an oxidation risk was present.

### Example 3-2

A sodium bisulfite was further added to the sodium bisulfite aqueous solution of Example 3-1 to which the deposit extracted liquid was added such that the sodium bisulfite concentration was 50 mg/L, and the measurement was performed for eight minutes in the same manner as in Example 3-1. The sodium bisulfite was further added such that the sodium bisulfite concentration was 100 mg/L, and the measurement was performed in the same manner. As a result, maximum values of the oxidation-reduction potentials in eight minutes were 344 mV and 329 mV, respectively. In any case, the hydrogen ion concentration (pH) of the sodium bisulfite aqueous solution was 2 and the solution temperature was 26° C. during the measurement of the change over time.

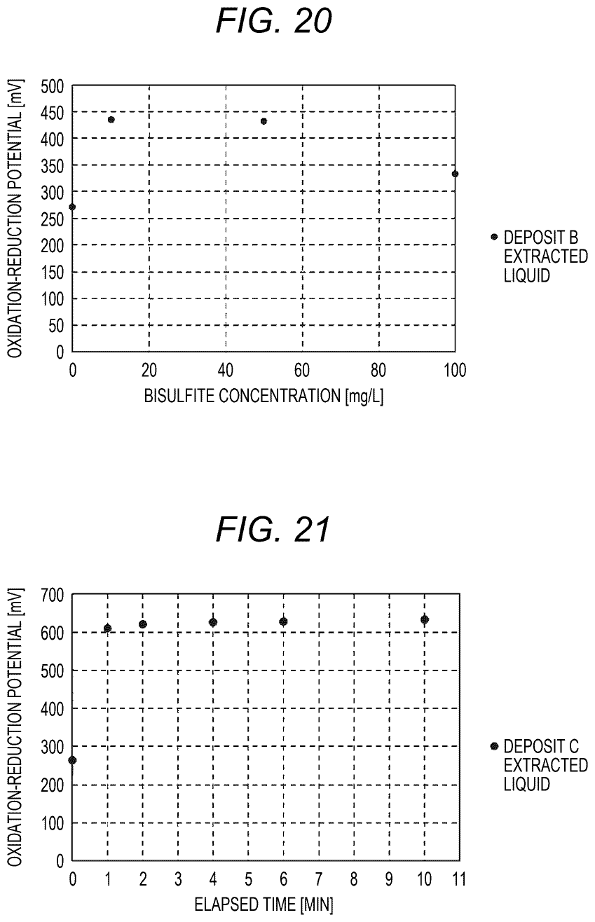

FIG. 20 shows a relation between a bisulfite (sodium bisulfite) concentration and the maximum value of the oxidation-reduction potential obtained from the above results.

From the above results, as shown in FIG. 20, the maximum value of the bisulfite concentrations was as large as 436 mV in the case of 10 mg/L of bisulfite, and this result was set as the oxidation potential of the deposit. This was the same as the oxidation potential obtained in Example 3-1, and it was determined that the oxidation potential of the deposit B was high, and the oxidation risk was present. On the other hand, it was determined that the oxidation risk was moderate in consideration of a matter that the oxidation potential was a value lower than 600 mV at which the oxidation risk was determined to be severe at pH 7, and that the oxidation-reduction potential is generally increased when the pH was low. In addition, in consideration of a change in pH before and after the addition of the deposit extracted liquid, when only the pH was changed without adding the deposit extracted liquid, the oxidation-reduction potential of the aqueous solution was equivalent to the value of the oxidation-reduction potential in 1 mol/L nitric acid of Example 3-1, and the numerical value was 338 mV, and when the deposit extracted liquid was used, the oxidation potential when the bisulfite concentration was 100 mg/L was equivalent to the numerical value, and thus it was estimated that the bisulfite concentration must be 100 mg/L to sufficiently reduce the oxidation risk of the deposit B.

### Example 4

In a water treatment plant C operated for about 2 months, since a RO module differential pressure increased and there was a concern that an oxidation risk occurred due to an increase in a deposit on a membrane surface, the oxidation risk was examined using an oxidation risk evaluation method performed by a catalytic reaction. A semipermeable membrane element SU-720 (manufactured by Toray Industries, Inc.) used in the plant was disassembled to take a separation membrane. A deposit was collected using a silicon jig from the separation membrane, and was added in a glass petri dish. The deposit was dried in a dryer set at 120° C. for 2 hours. The product is referred to as a deposit C.

To a sealable glass bottle, 20 mL of 1 mol/L nitric acid was added, and 4.0 g of the deposit C was further added, followed by sealing and standing at room temperature. After about 70 hours, the supernatant was collected in another glass bottle using a dropper. The product is referred to as a deposit extracted liquid C.

In order to prepare 400 mL of an aqueous solution having a sodium bisulfite concentration of 10 mg/L and a sodium chloride concentration of 32000 mg/L, sodium chloride, sodium bisulfite, pure water, and a stirrer bar were added in a 1 L beaker, followed by stirring with a magnetic stirrer to dissolve these. To this solution, 5 mL of the deposit extracted liquid C was added, and the oxidation-reduction potential was measured every one minute for 10 minutes using an oxidation-reduction potential meter while stirring to measure the change over time. A pH and a solution temperature were measured using a hydrogen ion concentration (pH) meter and an alcohol thermometer. The results are shown in FIG. 21. The maximum value of the oxidation-reduction potential was 633 mV. In this case, the pH was 2 and the solution temperature was 25° C.

From the results of the above and Example 3-1, it was determined that the oxidation potential of the deposit C was high and higher than the oxidation potential of the deposit B, and the oxidation risk was severe.

Although various embodiments have been described above, the present invention is not limited to these examples. It is apparent to those skilled in the art that various changes or modifications can be conceived within the scope described in the claims, and it should be naturally understood that those belong to the technical scope of the present invention. In addition, the components described in the above embodiments may be combined freely without departing from the gist of the invention.

According to the present invention, it is possible to simply and quickly evaluate an oxidation risk of a separation membrane, which is difficult to determine in the related art. In addition, since it is possible to easily and quickly know the oxidation risk and it is easier to identify a cause of deterioration or select a measure to be taken, reliable improvement of an operation situation of a water treatment plant is expected.

### REFERENCE SIGNS LIST

1: raw water storage tank
2: raw water feed pump
3: pretreatment membrane filtration unit
4: pretreatment membrane filtrate water storage tank
5: separation membrane filtration unit
6: booster pump
7: pressurizing pump
8: raw water pipe
9: pretreatment membrane filtrate water pipe
10: separation membrane filtration feed water pipe
11: separation membrane filtration permeate water pipe
12: separation membrane filtration concentrate water pipe
13: hose
14: flow meter
15: one-touch joint
16: water passing container opening and closing portion
17: flow control valve
18: water passing direction
19: water passing container
19a: unit water passing member
20: separation membrane

1. An oxidation risk evaluation method of a separation membrane in a water treatment plant, comprising:
collecting a deposit on a used separation membrane;
bringing the deposit into contact with a solution containing a sulfite or bisulfite; and
evaluating an oxidation potential of the deposit based on a generated oxidative substance.

2. The oxidation risk evaluation method of a separation membrane according to claim 1, wherein a deposit extracted liquid is used instead of the deposit.

3. The oxidation risk evaluation method of a separation membrane according to claim 1, wherein the separation membrane is any one of the following:
A: a separation membrane taken from a semipermeable membrane element that separates a feed water into a permeate water and a concentrate water in a water treatment plant,
B: a separation membrane provided inside a water passing member installed in a feed water line of a water treatment plant, and
C: a separation membrane provided inside a water passing member installed in a concentrate water line in a water treatment plant in which a semipermeable membrane element separating a feed water into a permeate water and a concentrate water is used.

4. The oxidation risk evaluation method of a separation membrane according to claim 1, wherein
the oxidation risk evaluation method of a separation membrane is an oxidation risk evaluation method performed by a catalytic reaction, and the method of evaluating the oxidation potential of the deposit is a method of measuring a change over time in an oxidation power index value of the solution brought into contact with the deposit, and any one of the following is evaluated:
A: a maximum value of the oxidation power index value,
B: a change rate of the oxidation power index value per unit time,
C: a maximum value of a moving average value of the oxidation power index value, and
D: a change rate of a moving average value of the oxidation power index value per unit time.

5. The oxidation risk evaluation method of a separation membrane according to claim 1, wherein
the method of evaluating the oxidation potential of the deposit is either one of the following A or B,
A: a method of adding a sulfite or bisulfite to the solution brought into contact with the deposit to change a sulfite or bisulfite concentration of the solution, and measuring an oxidation power index value when the deposit is brought into contact with the solution,
B: a method of using two or more types of the solutions containing a sulfite or bisulfite with different concentrations, and measuring the oxidation power index value of the solutions brought into contact with the deposit.

6. (canceled)

7. The oxidation risk evaluation method of a separation membrane according to claim 5, wherein
the method of evaluating the oxidation potential of the deposit is an evaluation of at least any one of the following:
A: a maximum value of the oxidation power index value, and
B: a change rate of the oxidation power index value per unit sulfite or bisulfite concentration.

8. The oxidation risk evaluation method of a separation membrane according to claim 5, wherein after the oxidation power index value becomes maximum, either of the following A or B is performed,
A: changing the concentration of the solution by further adding the sulfite or bisulfite to the solution, and measuring the oxidation power index value of the solution,
B: using further one or more solution containing the sulfite or bisulfite with a higher concentration to brought into contact with the deposit, and measuring the oxidation power index value of the solution.

9. (canceled)

10. The oxidation risk evaluation method of a separation membrane according to claim 8, wherein
the method of evaluating the oxidation potential of the deposit is a method of evaluating at least any one of the following:
A: a sulfite or bisulfite concentration at which the oxidation power index value is equal to or less than the oxidation power index value before contact with the deposit, and
B: a change rate of the oxidation power index value per unit sulfite or bisulfite concentration.

11. The oxidation risk evaluation method of a separation membrane according to claim 1, wherein
the deposit is brought into contact with the solution containing the sulfite or bisulfite having a hydrogen ion concentration index (pH) of 9.0 or more.

12. A non-transitory computer-readable recording medium storing an oxidation risk evaluation program of a separation membrane in a water treatment plant, which utilizes the oxidation risk evaluation method according to claim 1, wherein
in order to evaluate an oxidation risk, the program causes a computer to function as a data recording unit configured to record input data to the computer, and the program causes a computer to function as a means to evaluate the oxidation potential of the deposit based on the recorded data, and the input data includes a condition and an oxidation power index value when the deposit collected from the used separation membrane is brought into contact with the solution containing the sulfite or bisulfite.

13. The non-transitory computer-readable recording medium storing the oxidation risk evaluation program according to claim 12, wherein
the oxidation risk evaluation method of a water treatment plant is an oxidation risk evaluation method performed by a catalytic reaction, and the unit configured to evaluate the oxidation potential of the deposit is a unit configured to evaluate, based on a data over time of the oxidation power index value, at least any one of the following:
A: a maximum value of the oxidation power index value,
B: a change rate of the oxidation power index value per unit time,
C: a maximum value of a moving average value of the oxidation power index value, and
D: a change rate of a moving average value of the oxidation power index value per unit time.

14. The non-transitory computer-readable recording medium storing the oxidation risk evaluation program according to claim 12, wherein
the unit configured to evaluate the oxidation potential of the deposit is a unit configured to evaluate, based on a relation of the oxidation power index value to a sulfite or bisulfite concentration, any one of the following:
A: a maximum value of the oxidation power index value, and
B: a change rate of the oxidation power index value per unit sulfite or bisulfite concentration.

15. The non-transitory computer-readable recording medium storing the oxidation risk evaluation program according to claim 12, wherein
the unit configured to evaluate the oxidation potential of the deposit is a unit configured to evaluate, based on a relation of the oxidation power index value to a sulfite or bisulfite concentration after the oxidation power index value becomes maximum, any one of the following:
A: a sulfite or bisulfite concentration at which the oxidation power index value is equal to or less than the oxidation power index value before contact with the deposit, and
B: a change rate of the oxidation power index value per unit sulfite or bisulfite concentration.

16. (canceled)

17. An oxidation risk evaluation device of a separation membrane in a water treatment plant, the oxidation risk evaluation device comprising:
a data input unit configured to input, to a computer, a condition and an oxidation power index value when a deposit collected from a used separation membrane is brought into contact with a solution containing a sulfite or bisulfite;
a data recording unit configured to record the condition and the oxidation power index value in the computer; and
a unit configured to evaluate an oxidation potential by the method according to claim 1.
